(19) United States
(12) Patent Application Publication
(10) Pub. No.: US 2010/0330631 A1
(43) Pub. Date: Dec. 30, 2010

(54) Cis-aconitate Decarboxylase Mutants Having Improved Enzymatic Activity

(75) Inventors: Hsin-Ju Hsieh, Hsinchu City (TW); Pei-Ching Chang, Hsinchu County (TW); Kelly Teng, San Diego, CA (US)

Correspondence Address: OCCHIUTI ROHLICEK & TSAO, LLP, 10 FAWCETT STREET, CAMBRIDGE, MA, 02138, US

(73) Assignee: Industrial Technology Research Institute, Hsinchu (TW)

(21) Appl. No.: 12/494,487

(22) Filed: Jun. 30, 2009

(51) Int. Cl. C12P 7/46; C12N 9/14; C12N 15/11; C12N 15/00

(52) U.S. Cl. 435/145; 435/195; 536/23.2; 435/320.1

(57) ABSTRACT

Cis-aconitate decarboxylase mutants having one or more mutations in a C-terminal region as compared with a wild-type cis-aconitate decarboxylase of Aspergillus terreus.

### BACKGROUND OF THE INVENTION

Itaconic acid (IA), an essential compound used in manufacture of various products (e.g., acrylic fibers, rubbers, artificial diamonds, and lens), is highly demanded in the chemical industry. Certain microorganisms, such as Aspergillus terreus, produce this compound. It has been found that cis-aconitate decaroxylase (CAD) plays the key role in the biosynthesis of this compound.

### SUMMARY OF THE INVENTION

The present invention is based on an unexpected discovery that genetically modified Aspergillus stains containing CAD mutants that carry one or more mutations in a C-terminal region produce higher levels of IA as compared with wild-type Aspergillus.

Accordingly, one aspect of this invention features an isolated polypeptide having the amino acid sequence of a mutated CAD that has a mutation in the region corresponding to 441-490 (e.g., 461-490 or 481-490) of the amino acid sequence of a wild-type CAD (SEQ ID NO:1). The mutation can be located at the position corresponding to position 490 in SEQ ID NO:1. In one example, this mutation is substitution of a peptide fragment (e.g., GI or GIK) for V at position 490 in SEQ ID NO:1. The mutated CAD described above can further include a mutation at the position corresponding to position 489 in SEQ ID NO:1 (e.g., L at position 489 in SEQ ID NO:1 being replaced with F). Examples of the CAD mutants described herein include, but are not limited to, a polypeptide having the amino acid sequence of SEQ ID NO:3, 5, or 7.

Another aspect of this invention features a nucleic acid encoding any of the polypeptides disclosed above and host cells containing the nucleic acid, which is in operative linkage with a promoter functional in the host cell. A promoter sequence is a nucleotide sequence containing an element(s) necessary for initiating transcription of an operably linked nucleic acid sequence. At a minimum, a promoter contains an RNA polymerase binding site. It can further contain one or more enhancer elements that enhance transcription, or contain one or more regulatory elements that control the on/off status of the promoter.

Also within the scope of this invention is a method for producing IA by culturing the host cell disclosed above in a suitable medium to allow production of IA. The IA thus produced can be isolated from the culture medium.

The details of one or more embodiments of the invention are set forth in the description below. Other features or advantages of the present invention will be apparent from the following detailed description of several examples, and also from the appended claims.

### DETAILED DESCRIPTION OF THE INVENTION

Described herein is a mutated CAD that displays higher enzymatic activity relative to its wild-type counterpart.

CAD, which converts cis-aconitic acid to itaconic acid, is the key enzyme involved in the biosynthesis of IA. The term “CAD” used herein refers to any naturally occurring CADs (i.e., wild-type CAD). One example is the A. terreus CAD described in Dwiarti et al., J. Bioscience and Bioengineering, 94 (1):29-33, 2002 and WO 2009/014437). Provided below are the amino acid sequence (SEQ ID NO: 1) of this A. terreus CAD and an example of its encoding nucleotide sequence (SEQ ID NO:2):

| Amino Acid Sequence of A. terreus | |
|---|---|
| (SEQ ID NO: 1) | |
| atgaccaagcagtctgctgattccaacgcgaagtctggtgtgacctctgagatctgtcac | (SEQ ID NO: 2) |
| M T K Q S A D S N A K S G V T S E I C H | (SEQ ID NO: 1) |
| tgggcgtctaatctcgccactgatgatatcccgagcgacgttctggagcgtgcaaaatac | |
| W A S N L A T D D I P S D V L E R A K Y | |
| ctgatcctggatggtatcgcgtgcgcgtgggtaggtgctcgtgtcccatggtctgaaaaa | |
| L I L D G I A C A W V G A R V P W S E K | |
| tacgttcaagcgaccatgtctttcgaacctccgggtgcgtgtcgtgtcatcggttacggc | |
| Y V Q A T M S F E P P G A C R V I G Y G | |
| cagaaactgggtccggtagcggctgccatgacgaactctgcatttattcaggcgaccgaa | |
| Q K L G P V A A A M T N S A F I Q A T E | |
| ctcgatgactatcactctgaagcgccgctgcattccgcgtctatcgttctcccggcagtt | |
| L D D Y H S E A P L H S A S I V L P A V | |
| ttcgcggcgagcgaagtactggccgaacagggtaaaaccatctctggtattgacgtgatt | |
| F A A S E V L A E Q G K T I S G I D V I | |
| ctggctgcgatcgttggtttcgagagcggtcctcgcatcggcaaagcgatctacggttct | |
| L A A I V G F E S G P R I G K A I Y G S | |
| gacctcctgaacaacggctggcactgcggtgcggtatatggcgcaccggctggtgcgctc | |
| D L L N N G W H C G A V Y G A P A G A L | |
| gcaactggtaagctcctgggcctcacgccggacagcatggaagatgcactgggtattgcc | |
| A T G K L L G L T P D S M E D A L G I A | |
| tgcacgcaagcatgcggcctcatgtccgcgcagtatggtggcatggttaaacgtgttcag | |
| C T Q A C G L M S A Q Y G G M V K R V Q | |
| cacggtttcgcagcgcgtaatggtctcctcggtggcctcctggctcacggcggctacgag | |
| H G F A A R N G L L G G L L A H G G Y E | |
| gcgatgaaaggtgttctcgagcgttcttacggtggcttcctgaagatgttcaccaagggc | |
| A M K G V L E R S Y G G F L K M F T K G | |
| aacggtcgtgaaccgccgtacaaagaagaagaggttgtggctggtctgggtagcttctgg | |
| N G R E P P Y K E E E V V A G L G S F W | |
| cacaccttcaccattcgtatcaaactgtacgcgtgctgcggtctcgtacacggtcctgtt | |
| H T F T I R I K L Y A C C G L V H G P V | |
| gaagccattgaaaacctccagggtcgttacccggaactgctcaatcgtgctaacctgtct | |
| E A I E N L Q G R Y P E L L N R A N L S | |
| aacatccgccacgttcacgtacaactctctaccgcgagcaactcccactgtggttggatc | |
| N I R H V H V Q L S T A S N S H C G W I | |
| ccagaagagcgcccaatctcttctatcgcgggtcaaatgtctgtcgcatatatcctcgcc | |
| P E E R P I S S I A G Q M S V A Y I L A | |
| gttcagctcgttgaccaacagtgtctgctcagccagttctccgagtttgacgataatctg | |
| V Q L V D Q Q C L L S Q F S E F D D N L | |
| gaacgcccggaagtgtgggacctggcacgtaaggttaccagctctcaatctgaggagttc | |
| E R P E V W D L A R K V T S S Q S E E F | |
| gaccaggacggtaactgtctctctgccggtcgcgtccgtattgagttcaacgacggctcc | |
| D Q D G N C L S A G R V R I E F N D G S | |
| tccatcaccgaatccgttgagaagccgctcggtgtaaaggaaccaatgccaaatgaacgc | |
| S I T E S V E K P L G V K E P M P N E R | |
| atcctgcacaaataccgtaccctggcgggttctgtaacggacgaaagccgtgttaaggag | |
| I L H K Y R T L A G S V T D E S R V K E | |
| atcgaggatctcgtgctcggcctggaccgtctgaccgatattagcccgctcctcgagctg | |
| I E D L V L G L D R L T D I S P L L E L | |
| ctgaattgtccggttaaatccccactggtttaa | |
| L N C P V K S P L V - | |

Other natural CADs are polypeptides found in non-A. terreus species that possess the same enzymatic activity. These polypeptides are highly homologous to the A. terreus CAD described above, i.e., having an amino acid sequence at least 75% (85%, 90%, or 95%) identical to SEQ ID NO:1. The amino acid sequences and their encoding gene sequences can be retrieved from gene/protein databases, e.g., GenBank, using SEQ ID NOs:1 and 2 as queries.

The percent identity of two amino acid sequences is determined using the algorithm of Karlin and Altschul Proc. Natl. Acad. Sci. USA 87:2264-68, 1990, as modified in Karlin and Altschul Proc. Natl. Acad. Sci. USA 90:5873-77, 1993. Such an algorithm is incorporated into the BLASTN and BLASTX programs (version 2.0) of Altschul, et al. J. Mol. Biol. 215:403-10, 1990. BLAST protein searches can be performed with the BLASTX program, score=50, wordlength=3 to obtain amino acid sequences homologous to the protein molecules of the invention. Where gaps exist between two sequences, Gapped BLAST can be utilized as described in Altschul et al., Nucleic Acids Res. 25:3389-3402, 1997. When utilizing BLAST and Gapped BLAST programs, the default parameters of the respective programs (e.g., BLASTX and BLASTN) can be used.

The CAD mutant of this invention contains one or more mutations in the region of a naturally-occurring CAD that corresponds to 441-490 in SEQ ID NO:1. The one or more mutations can be deletion, insertion, or amino acid residue substitution (e.g., a fragment being replaced with a single amino acid residue or a single residue being replaced with a fragment). Listed below are a number of exemplary CAD mutants, including their amino acid sequences (mutated positions bold-faced) and encoding nucleotide sequences:

| | |
|---|---|
| mCAD 1: | |
| atgaccaaacaatctgcggacagcaacgcaaagtcaggagttacgtccgaaatatgtcat | (SEQ ID NO: 4) |
| M T K Q S A D S N A K S G V T S E I C H | (SEQ ID NO: 3) |
| tgggcatccaacctggccactgacgacatcccttcggacgtattagaaagagcaaaatac | |
| W A S N L A T D D I P S D V L E R A K Y | |
| cttattctcgacggtattgcatgtgcctgggttggtgcaagagtgccttggtcagagaag | |
| L I L D G I A C A W V G A R V P W S E K | |
| tatgttcaggcaacgatgagctttgagccgccgggggcctgcagggtgattggatatgga | |
| Y V Q A T M S F E P P G A C R V I G Y G | |
| cagaaactggggcctgttgcagcagccatgaccaattccgctttcatacaggctacggag | |
| Q K L G P V A A A M T N S A F I Q A T E | |
| cttgacgactaccacagcgaagcccccctacactctgcaagcattgtccttcctgcggtc | |
| L D D Y H S E A P L H S A S I V L P A V | |
| tttgcagcaagtgaggtcttagccgagcagggcaaaacaatttccggtatagatgttatt | |
| F A A S E V L A E Q G K T I S G I D V I | |
| ctagccgccattgtggggtttgaatctggcccacggatcggcaaagcaatctacggatcg | |
| L A A I V G F E S G P R I G K A I Y G S | |
| gacctcttgaacaacggctggcattgtggagctgtgtatggcgctccagccggtgcgctg | |
| D L L N N G W H C G A V Y G A P A G A L | |
| gccacaggaaagctcctcggtctaactccagactccatggaagatgctctcggaattgcg | |
| A T G K L L G L T P D S M E D A L G I A | |
| tgcacgcaagcctgtggtttaatgtcggcgcaatacggaggcatggtaaagcgtgtgcaa | |
| C T Q A C G L M S A Q Y G G M V K R V Q | |
| cacggattcgcagcgcgtaatggtcttcttgggggactgttggcccatggtgggtacgag | |
| H G F A A R N G L L G G L L A H G G Y E | |
| gcaatgaaaggtgtcctggagagatcttacggcggtttcctcaagatgttcaccaagggc | |
| A M K G V L E R S Y G G F L K M F T K G | |
| aacggcagagagcctccctacaaagaggaggaagtggtggctggtctcggttcattctgg | |
| N G R E P P Y K E E E V V A G L G S F W | |
| catacctttactattcgcatcaagctctatgcctgctgcggacttgtccatggtccagtc | |
| H T F T I R I K L Y A C C G L V H G P V | |
| gaggctatcgaaaaccttcaggggagataccccgagctcttgaatagagccaacctcagc | |
| E A I E N L Q G R Y P E L L N R A N L S | |
| aacattcgccatgttcatgtacagctttcaacggcctcgaacagtcactgtggatggata | |
| N I R H V H V Q L S T A S N S H C G W I | |
| ccagaggagagacccatcagttcaatcgcagggcagatgagtgtcgcatacattctcgcc | |
| P E E R P S I S S I A G Q M S V A Y L A | |
| gtccagctggtcgaccagcaatgtcttttgtcccagttttctgagtttgatgacaacctg | |
| V W L V D Q Q C L L S Q F S E F D D N L | |
| gagaggccagaagtttgggatctggccaggaaggttacttcatctcaaagcgaagagttt | |
| E R P E V W D L A R K V T S S Q S E E F | |
| gatcaagacggcaactgtctcagtgcgggtcgcgtgaggattgagttcaacgatggttct | |
| D Q D G N C L S A G R V R I E F N D G S | |
| tctattacggaaagtgtcgagaagcctcttggtgtcaaagagcccatgccaaacgaacgg | |
| S I T E S V E K P L G V K E P M P N E R | |
| attctccacaaataccgaacccttgctggtagcgtgacggacgaatcccgggtgaaagag | |
| I L H K Y R T L A G S V T D E S R V K E | |
| attgaggatcttgtcctcggcctggacaggctcaccgacattagcccattgctggagctg | |
| I E D L V L G L D R L T D I S P L L E L | |
| Ctgaattgccccgtgaaatcgccccttggtatataa | |
| L N C P V K S P L G I - | |
| mCAD 2 | |
| atgaccaaacaatctgcggacagcaacgcaaagtcaggagttacgtccgaaatatgtcat | (SEQ ID NO: 6) |
| M T K Q S A D S N A K S G V T S E I C H | (SEQ ID NO: 5) |
| tgggcatccaacctggccactgacgacatcccttcggacgtattagaaagagcaaaatac | |
| W A S N L A T D D I P S D V L E R A K Y | |
| cttattctcgacggtattgcatgtgcctgggttggtgcaagagtgccttggtcagagaag | |
| L I L D G I A C A W V G A R V P W S E K | |
| tatgttcaggcaacgatgagctttgagccgccgggggcctgcagggtgattggatatgga | |
| Y V Q A T M S F E P P G A C R V I G Y G | |
| cagaaactggggcctgttgcagcagccatgaccaattccgctttcatacaggctacggag | |
| Q K L G P V A A A M T S N A F I Q A T E | |
| cttgacgactaccacagcgaagcccccctacactctgcaagcattgtccttcctgcggtc | |
| L D D Y H S E A P L H S A S I V L P A V | |
| tttgcagcaagtgaggtcttagccgagcagggcaaaacaatttccggtatagatgttatt | |
| F A A S E V L A E Q G K T I S G I D V I | |
| ctagccgccattgtggggtttgaatctggcccacggatcggcaaagcaatctacggatcg | |
| L A A I V G F E S G P R I G K A I Y G S | |
| gacctcttgaacaacggctggcattgtggagctgtgtatggcgctccagccggtgcgctg | |
| D L L N N G W H C G A V Y G A P A G A L | |
| gccacaggaaagctcctcggtctaactccagactccatggaagatgctctcggaattgcg | |
| A T G K L L G L T P D S M E D A L G I A | |
| tgcacgcaagcctgtggtttaatgtcggcgcaatacggaggcatggtaaagcgtgtgcaa | |
| C T Q A C G L M S A Q Y G G M V K R V Q | |
| cacggattcgcagcgcgtaatggtcttcttgggggactgttggcccatggtgggtacgag | |
| H G F A A R N G L L G G L L A H G G Y E | |
| gcaatgaaaggtgtcctggagagatcttacggcggtttcctcaagatgttcaccaagggc | |
| A M K G V L E R S Y G G F L K M F T K G | |
| aacggcagagagcctccctacaaagaggaggaagtggtggctggtctcggttcattctgg | |
| N G R E P P Y K E E E V V A G L G S F W | |
| catacctttactattcgcatcaagctctatgcctgctgcggacttgtccatggtccagtc | |
| H T F T I R I K L Y A C C G L V H G P V | |
| gaggctatcgaaaaccttcaggggagataccccgagctcttgaatagagccaacctcagc | |
| E A I E N L Q G R Y P E L L N R A N L S | |
| aacattcgccatgttcatgtacagctttcaacggcctcgaacagtcactgtggatggata | |
| N I R H V H V Q L S T A S N S H C G W I | |
| ccagaggagagacccatcagttcaatcgcagggcagatgagtgtcgcatacattctcgcc | |
| P E E R P I S S I A G Q M S V A Y I L A | |
| gtccagctggtcgaccagcaatgtcttttgtcccagttttctgagtttgatgacaacctg | |
| V Q L V D Q Q C L L S Q F S E F D D N L | |
| gagaggccagaagtttgggatctggccaggaaggttacttcatctcaaagcgaagagttt | |
| E R P E V W D L A R K V T S S Q S E E F | |
| gatcaagacggcaactgtctcagtgcgggtcgcgtgaggattgagttcaacgatggttct | |
| D Q D G N C L S A G R V R I E F N D G S | |
| tctattacggaaagtgtcgagaagcctcttggtgtcaaagagcccatgccaaacgaacgg | |
| S I T E S V E K P L G V K E P M P N E R | |
| attctccacaaataccgaacccttgctggtagcgtgacggacgaatcccgggtgaaagag | |
| I L H K Y R T L A G S V T D E S R V K E | |
| attgaggatcttgtcctcggcctggacaggctcaccgacattagcccattgctggagctg | |
| I E D L V L G L D R L T D I S P L L E L | |
| Ctgaattgccccgtgaaatcgcccctgggtataaaataa | |
| L N C P V K S P L G I K - | |
| mCAD 3: | |
| atgaccaaacaatctgcggacagcaacgcaaagtcaggagttacgtccgaaatatgtcat | (SEQ ID NO: 8) |
| M T K Q S A D S N A K S G V T S E I C H | (SEQ ID NO: 7) |
| tgggcatccaacctggccactgacgacatcccttcggacgtattagaaagagcaaaatac | |
| W A S N L A T D D I P S D V L E R A K Y | |
| cttattctcgacggtattgcatgtgcctgggttggtgcaagagtgccttggtcagagaag | |
| L I L D G I A C A W V G A R V P W S E K | |
| tatgttcaggcaacgatgagctttgagccgccgggggcctgcagggtgattggatatgga | |
| Y V Q A T M S F E P P G A C R V I G Y G | |
| cagaaactggggcctgttgcagcagccatgaccaattccgctttcatacaggctacggag | |
| Q K L G P V A A A M T S N A F I Q A T E | |
| cttgacgactaccacagcgaagcccccctacactctgcaagcattgtccttcctgcggtc | |
| L D D Y H S E A P L H S A S I V L P A V | |
| tttgcagcaagtgaggtcttagccgagcagggcaaaacaatttccggtatagatgttatt | |
| F A A S E V L A E Q G K T I S G I D V I | |
| ctagccgccattgtggggtttgaatctggcccacggatcggcaaagcaatctacggatcg | |
| L A A I V G F E S G P R I G K A I Y G S | |
| gacctcttgaacaacggctggcattgtggagctgtgtatggcgctccagccggtgcgctg | |
| D L L N N G W H C G A V Y G A P A G A L | |
| gccacaggaaagctcctcggtctaactccagactccatggaagatgctctcggaattgcg | |
| A T G K L L G L T P D S M E D A L G I A | |
| tgcacgcaagcctgtggtttaatgtcggcgcaatacggaggcatggtaaagcgtgtgcaa | |
| C T Q A C G L M S A Q Y G G M V K R V Q | |
| cacggattcgcagcgcgtaatggtcttcttgggggactgttggcccatggtgggtacgag | |
| H G F A A R N G L L G G L L A H G G Y E | |
| gcaatgaaaggtgtcctggagagatcttacggcggtttcctcaagatgttcaccaagggc | |
| A M K G V L E R S Y G G F L K M F T K G | |
| aacggcagagagcctccctacaaagaggaggaagtggtggctggtctcggttcattctgg | |
| N G R E P P Y K E E E V V A G L G S F W | |
| catacctttactattcgcatcaagctctatgcctgctgcggacttgtccatggtccagtc | |
| H T F T I R I K L Y A C C G L V H G P V | |
| gaggctatcgaaaaccttcaggggagataccccgagctcttgaatagagccaacctcagc | |
| E A I E N L Q G R Y P E L L N R A N L S | |
| aacattcgccatgttcatgtacagctttcaacggcctcgaacagtcactgtggatggata | |
| N I R H V H V Q L S T A S N S H V G W I | |
| ccagaggagagacccatcagttcaatcgcagggcagatgagtgtcgcatacattctcgcc | |
| P E E R P I S S I A G Q M S V A Y I L A | |
| gtccagctggtcgaccagcaatgtcttttgtcccagttttctgagtttgatgacaacctg | |
| V Q L V D Q Q C L L S Q F S E F D D N L | |
| gagaggccagaagtttgggatctggccaggaaggttacttcatctcaaagcgaagagttt | |
| E R P E V W D L A R K V T S S Q S E E F | |
| gatcaagacggcaactgtctcagtgcgggtcgcgtgaggattgagttcaacgatggttct | |
| D Q D G N C L S A G R V R I E F N D G S | |
| tctattacggaaagtgtcgagaagcctcttggtgtcaaagagcccatgccaaacgaacgg | |
| S I T E S V E K P L G V K E P M P N E R | |
| attctccacaaataccgaacccttgctggtagcgtgacggacgaatcccgggtgaaagag | |
| I L H K Y R T L A G S V T D E S R V K E | |
| attgaggatcttgtcctcggcctggacaggctcaccgacattagcccattgctggagctg | |
| I E D L V L G L D R L T D I S P L L E L | |
| Ctgaattgccccgtgaaatcgccctttggtatataa | |
| L N C P V K S P F G I - | |

Any of the above-mentioned CAD mutants can be prepared by conventional recombinant technology. For example, one or more mutations can be introduced into a nucleotide sequence encoding a wild-type CAD and the mutated encoding sequence can be expressed in a suitable host cell to produce the mutated CAD polypeptide. The increased enzymatic activity of the polypeptide can be confirmed by conventional methods. In one example, the IA level produced in the host cells can be determined by HPLC and an elevated level of IA production relative to the same type of host cells expressing a wild-type CAD indicates that the mutant CAD possesses improved enzymatic activity as compared with the wild-type CAD.

After its elevated enzymatic activity has been confirmed, the CAD mutant can be used for producing IA. More specifically, its coding sequence can be cloned into a suitable expression plasmid and introduced into a suitable host microorganism. The microorganism is then cultured in a suitable medium for itaconic acid production. Preferably, the medium contains glucose or citrate as the precursor for making itaconic acid. After a sufficient culturing period, the medium is collected and the secreted itaconic acid is isolated.

Without further elaboration, it is believed that one skilled in the art can, based on the above description, utilize the present invention to its fullest extent. The following specific example is, therefore, to be construed as merely illustrative, and not limitative of the remainder of the disclosure in any way whatsoever. All publications cited herein are incorporated by reference.

Itaconic Acid Production in A. terreus Strains Containing Mutated CADs
Generating A. terreus Mutants/Fusants Containing Mutated CADs

Mutants of A. terreus were generated by random mutagenesis as follows. A. terreus ATCC10020 cells at a concentration of 10⁸ cell/ml was incubated overnight in an IA producing medium (pH 2.8) that contains, in one liter distilled water, 100 g glucose, 6 g (NH₄)₂SO₄, 0.2 g KH₂PO₄, 1 g MgSO₄.7H₂O, 0.4 g CaSO₄, 0.5×10⁻³ g CuSO4, 0.5×10⁻³ g ZnSO4.4H2O, 3×10⁻³ g FeCl₃. The cells were collected, washed, and then incubated with 10-15 g/l Lysosome (contains beta-glucanase, cellulose, protease and chitinase) and a mutagen, i.e., 1-5 g/l 1-methyl-3-nitro-nitrosoguanidine (NTG, 1-5 g/l) or ethylmethanesulfonate (EMS, 2-200 mg/l), to induce mutations. The cells were again collected and re-suspended in a medium, placed on a 2-deoxy-glucose (2-DG)-potato agar plate (containing 0.25-5.0 g/l 2-DG), and cultured under suitable conditions to allow colony formation. Around 12.5-99.3% (3×10⁷-9×10⁸ CFU/ml) of the cells died in the presence of either mutagen. Each of the surviving colonies were cultured in 25 ml of the above-described IA production medium at a concentration of 10¹⁰ cell/ml and then plated on an IA-potato dextrose agar plate containing a pH indicator (pH 3.0-5.4), which was selected from bromocresol green, bromophenol blue, or congo red (30-130 mg/L). Colonies producing high levels of IA were selected based on their color shown on the plate.

Mutants M1-M5 were selected in the just-described screening process as producing high levels of IA. These mutants were then cultured in the IA-production medium for 3.5 days and the levels of IA in the culture media, filtered through a membrane having a pore size of 0.22 μm, were analyzed by HPLC, using the LiChroCART column (5-mm particle size, 125-mm length, 4-mm diameter, E. Merck, Germany). IA was eluted at 30° C. with a buffer containing 20 mM ortho-phosphoric acid at a flow rate of 1 ml/min. The eluted IA was detected at 230 nm with a Shimadzu SD-20A Absorbance Detector (Shimadzu, Japan). As shown in Table 1 below, mutants M1-M5 produced higher levels of IA than wild-type A. terreus.

TABLE 1
Itaconic Acid Production by M1-M5

| | IA concentration | IA concentration | Yield | Yield |
|---|---|---|---|---|
| Strains | (g/l) | (folds) | (%) | (folds) |
| WT | 10.87 | 1 | 23.38 | 1 |
| M1 | 17.43 | 1.60 | 28.30 | 1.21 |
| M2 | 17.12 | 1.57 | 29.06 | 1.24 |
| M3 | 21.68 | 1.99 | 33.55 | 1.44 |
| M4 | 28.04 | 2.58 | 36.85 | 1.58 |
| M5 | 23.51 | 2.16 | 39.32 | 1.68 |

A. terreus mutants were also generated by genome shuffling with A. niger as follows. Aspergillus terreus ATCC10020 cells (10⁸ cell/ml) and Aspergillus niger NRRRL330 cells (10⁸ cell/ml) were cultured separately overnight at 35° C. in a medium (pH 3) containing, in one liter distilled water, 1500 g glucose, 2.5 g (NH₄)₂SO₄, 2 g KH₂PO₄, 0.5 g MgSO₄, 0.06×10⁻¹ g CuSO₄.5H₂O , 0.1×10⁻³ g ZnSO₄.7H2O, 0.1×10⁻³ g FeSO₄.24H₂O. All cells were collected, washed with a phosphate buffer (pH 6) containing 0.7 M KCl, and incubated in Lysosome for 2 hours with gentle shaking. The protoplasts thus released were subjected to osmosis using 1.4 M sorbitol and then collected by centrifugation at 1800 g. The protoplasts were re-suspended in distilled water to form crude protoplast suspensions. Equal volumes of A. terreus and A. niger crude protoplast suspensions were mixed and centrifuged at 1000 g, 4° C. for 10 min. The protoplast pellet thus formed was resuspended in a 0.05 M glycin-NaOH buffer (pH7.5) containing 30% (w/v) polyethylene glycol (PEG) 6000, 100 mM CaCl₂, and 0.7 M KCl. The suspension was incubated at 30° C. for 20 min, plated onto the surface of a potato dextrose agar plate, and incubated at 30° C. for 5-7 days to allow formation of colonies, which were fusants. The fusion frequency in this study was found to be ˜200 CFU/ml. Each of the fusants was then cultured in 25 ml of the IA production medium described above at 35° C. for 5.5 days in a rotary shaker (150 rev/min). Fusants G1-G6 were found to produce higher levels of IA than wild-type A. terreus.

TABLE 2
Itaconic Acid Production by G1-G6

| | IA concentration | IA concentration | Yield | Yield |
|---|---|---|---|---|
| Strains | (g/l) | (folds) | (%) | (folds) |
| WT | 21.83 | 1 | 27.20 | 1 |
| G1 | 35.97 | 1.65 | 49.69 | 1.83 |
| G2 | 35.63 | 1.63 | 50.26 | 1.85 |
| G3 | 29.33 | 1.34 | 67.12 | 2.47 |
| G4 | 31.90 | 1.46 | 50.46 | 1.86 |
| G5 | 31.62 | 1.45 | 55.29 | 2.03 |
| G6 | 30.98 | 1.42 | 53.69 | 1.97 |

### Determining Nucleotide Sequences Encoding CAD Mutants

The CAD genomic sequences of certain mutants/fusants were determined by conventional methods. Briefly, genomic DNAs and total RNAs were isolated from the mutants/fusants of interest using Wizard® Genomic DNA Purification kit (Promega, USA) and Epicentre® MasterPure™ RNA Purification kit (Biotechnologies, USA), respectively. A DNA fragment containing the CAD gene was amplified from the genomic DNAs by polymerase chain reaction (PCR) with tag polymerase (Invitrogen™, US) using the primers listed below:

| | |
|---|---|
| CAD1 (forward): | 5′-CAGCCATGACCAATTCCGCTTTCA-3′ |
| CAD1 (reverse): | 5′-AAGACCTCACTTGCTGCAAAGACC-3′ |
| Cad-f-2 (504-523): | 5′-TTGTGGAGCTGTGTATGGCG-3′ |
| Cad-(700-716)-F: | 5′-GTTGGCCCATGGTGGG-3′ |
| Cad-(251-270)-R: | 5′-CATGGCTGGETGCAACAGGCC-3′ |

The PCR conditions were: 94° C. for 5 s, 30 cycles of 94° C. for 20 s, 56° C. for 30 s, 72° C. for 2 min, and a final extension at 72° C. for 10 s.

The CAD coding sequences were also amplified by reverse transcription polymerase chain reaction (RT-PCR) using the total RNAs mentioned above as the template and the primers listed above with the Verso 1-step RT-PCR kit (AB gene, US) under the following conditions: 47° C. for 30 s, 94° C. for 2 min, 30 cycles of 94° C. for 20 s, 55° C. for 30 s, 72° C. for 2 min, and a final extension at 72° C. for 5 min.

Results obtained from this study indicate that M1 and M4 contain mCAD 1, G2 and G4 contain mCAD 2, and G3 contains mCAD 3. The amino acid sequences and nucleotide sequences of these CAD mutants are shown in pages 4-7 above.

### Determining Enzymatic Activity of CAD Mutants

The CAD activity of these mutated CAD were examined following the method described in Dwiarti et al., J. Bioscience and Bioengineering 94:29-33 (2002) with slight modifications. Briefly, A. terreus strains containing these CAD mutants were cultured in a suitable medium (10¹⁰ cells/ml) for 3 days at 35° C. in a rotary shaker (150 rev/min). The cultured cells were 20 harvested, suspended in a sodium phosphate buffer (0.2 M, pH 6.2) at a volume ratio of 1:10, and lyzed by pulsed sonication for 10 min to form crude lysates. The lysates were centrifuged and the resultant supernatants were collected. 4.5 ml of each supernatant were incubated with 0.5 ml of a solution containing cis-aconitic acid at a final concentration of 5.0 mM for 10 min at 40° C. The enzyme reaction was terminated by addition of 0.1 ml 12 M HCl into the reaction mixture. The amount of itaconic acid, the product of the enzymatic reaction, was measured by HPLC (column: LiChroCART, detector: SPD-20AD, 20 mM ortho-phosphoric acid, at 30° C., at 230 nm).

TABLE 4
CAD activity in A. terreus Strains Containing Mutated CADs

| | CAD activity | CAD activity | |
|---|---|---|---|
| Strains | (umol/mg/min) | (folds) | CAD |
| WT | 0.007 | 1 | WT |
| M1 | 0.056 | 8 | mCAD 1 |
| M4 | 0.064 | 9.1 | mCAD 1 |
| G2 | 0.009 | 1.3 | mCAD 2 |
| G3 | 0.008 | 1.1 | mCAD 3 |
| G4 | 0.018 | 2.6 | mCAD 2 |

As shown in Table 4 above, A. terreus strains containing the mutated CADs all exhibited increased CAD activity as compared with wild-type A. terreus.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. An isolated polypeptide, comprising the amino acid sequence of a mutated cis-aconitate decarboxylase (CAD), wherein the mutated CAD has a mutation in the region corresponding to 441-490 in SEQ ID NO:1 as compared to its wild-type counterpart.

2. The isolated polypeptide of claim 1, wherein the mutation is in the region corresponding to 461-490 in SEQ ID NO:1.

3. The isolated polypeptide of claim 2, wherein the mutation is in the region corresponding to 481-490 in SEQ ID NO:1.

4. The isolated polypeptide of claim 3, wherein the mutation is at the position corresponding to position 490 in SEQ ID NO:1.

5. The isolated polypeptide of claim 4, wherein the mutation is a substitution of a peptide fragment for V at position 490 in SEQ ID NO:1.

6. The isolated polypeptide of claim 5, wherein the peptide fragment is GI.

7. The isolated polypeptide of claim 6, wherein the polypeptide includes the amino acid sequence of SEQ ID NO:3.

8. The isolated polypeptide of claim 5, wherein the peptide fragment is GIK.

9. The isolated polypeptide of claim 8, wherein the polypeptide includes the amino acid sequence of SEQ ID NO:5.

10. The isolated polypeptide of claim 5, wherein the polypeptide further includes a mutation at the position corresponding to position 489 in SEQ ID NO:1.

11. The isolated polypeptide of claim 6, wherein the polypeptide further includes a mutation at the position corresponding to position 489 in SEQ ID NO:1.

12. The isolated polypeptide of claim 11, wherein the mutation is a substitution of F for L at position 489 in SEQ ID NO:1.

13. The isolated polypeptide of claim 12, wherein the polypeptide has the amino acid sequence of SEQ ID NO:7.

14. An isolated nucleic acid, comprising a nucleotide sequence encoding the polypeptide of claim 1.

15. The isolated nucleic acid of claim 14, wherein the nucleotide sequence encodes the amino acid sequence selected from the group consisting of SEQ ID NOs:3, 5, and 7.

16. The nucleic acid of claim 14, wherein the nucleic acid is an expression vector, in which the nucleotide sequence is operably linked to a promoter sequence.

17. The nucleic acid of claim 15, wherein the nucleic acid is an expression vector, in which the nucleotide sequence is operably linked to a promoter sequence.

18. A method for producing itaconic acid, comprising:
providing a host cell containing an expression cassette, in which a nucleotide sequence encoding the polypeptide of claim 1 is operably linked to a promoter,
culturing the host cell in a medium to allow expression of the polypeptide, thereby producing itaconic acid in the host cell.

19. The method of claim 16, wherein the polypeptide has the amino acid sequence selected from the group consisting of SEQ ID NOs:3, 5, and 7.

20. The method of claim 18, further comprising, after the culturing step, isolating the itaconic acid thus produced from the medium.


SEQUENCE LISTING (13 sequences)

SEQ ID NO: 1 - protein, 490 aa, Aspergillus terreus
MTKQSADSNAKSGVTSEICHWASNLATDDIPSDVLERAKYLILDGIACAWVGARVPWSEKYVQATMSFEPPGACRVIGYGQKLGPVAAAMTNSAFIQATELDDYHSEAPLHSASIVLPAVFAASEVLAEQGKTISGIDVILAAIVGFESGPRIGKAIYGSDLLNNGWHCGAVYGAPAGALATGKLLGLTPDSMEDALGIACTQACGLMSAQYGGMVKRVQHGFAARNGLLGGLLAHGGYEAMKGVLERSYGGFLKMFTKGNGREPPYKEEEVVAGLGSFWHTFTIRIKLYACCGLVHGPVEAIENLQGRYPELLNRANLSNIRHVHVQLSTASNSHCGWIPEERPISSIAGQMSVAYILAVQLVDQQCLLSQFSEFDDNLERPEVWDLARKVTSSQSEEFDQDGNCLSAGRVRIEFNDGSSITESVEKPLGVKEPMPNERILHKYRTLAGSVTDESRVKEIEDLVLGLDRLTDISPLLELLNCPVKSPLV

SEQ ID NO: 2 - DNA, 1473 bp, Aspergillus terreus
atgaccaagcagtctgctgattccaacgcgaagtctggtgtgacctctgagatctgtcactgggcgtctaatctcgccactgatgatatcccgagcgacgttctggagcgtgcaaaatacctgatcctggatggtatcgcgtgcgcgtgggtaggtgctcgtgtcccatggtctgaaaaatacgttcaagcgaccatgtctttcgaacctccgggtgcgtgtcgtgtcatcggttacggccagaaactgggtccggtagcggctgccatgacgaactctgcatttattcaggcgaccgaactcgatgactatcactctgaagcgccgctgcattccgcgtctatcgttctcccggcagttttcgcggcgagcgaagtactggccgaacagggtaaaaccatctctggtattgacgtgattctggctgcgatcgttggtttcgagagcggtcctcgcatcggcaaagcgatctacggttctgacctcctgaacaacggctggcactgcggtgcggtatatggcgcaccggctggtgcgctcgcaactggtaagctcctgggcctcacgccggacagcatggaagatgcactgggtattgcctgcacgcaagcatgcggcctcatgtccgcgcagtatggtggcatggttaaacgtgttcagcacggtttcgcagcgcgtaatggtctcctcggtggcctcctggctcacggcggctacgaggcgatgaaaggtgttctcgagcgttcttacggtggcttcctgaagatgttcaccaagggcaacggtcgtgaaccgccgtacaaagaagaagaggttgtggctggtctgggtagcttctggcacaccttcaccattcgtatcaaactgtacgcgtgctgcggtctcgtacacggtcctgttgaagccattgaaaacctccagggtcgttacccggaactgctcaatcgtgctaacctgtctaacatccgccacgttcacgtacaactctctaccgcgagcaactcccactgtggttggatcccagaagagcgcccaatctcttctatcgcgggtcaaatgtctgtcgcatatatcctcgccgttcagctcgttgaccaacagtgtctgctcagccagttctccgagtttgacgataatctggaacgcccggaagtgtgggacctggcacgtaaggttaccagctctcaatctgaggagttcgaccaggacggtaactgtctctctgccggtcgcgtccgtattgagttcaacgacggctcctccatcaccgaatccgttgagaagccgctcggtgtaaaggaaccaatgccaaatgaacgcatcctgcacaaataccgtaccctggcgggttctgtaacggacgaaagccgtgttaaggagatcgaggatctcgtgctcggcctggaccgtctgaccgatattagcccgctcctcgagctgctgaattgtccggttaaatccccactggtttaa

SEQ ID NO: 3 - protein, 491 aa, Aspergillus terreus
MTKQSADSNAKSGVTSEICHWASNLATDDIPSDVLERAKYLILDGIACAWVGARVPWSEKYVQATMSFEPPGACRVIGYGQKLGPVAAAMTNSAFIQATELDDYHSEAPLHSASIVLPAVFAASEVLAEQGKTISGIDVILAAIVGFESGPRIGKAIYGSDLLNNGWHCGAVYGAPAGALATGKLLGLTPDSMEDALGIACTQACGLMSAQYGGMVKRVQHGFAARNGLLGGLLAHGGYEAMKGVLERSYGGFLKMFTKGNGREPPYKEEEVVAGLGSFWHTFTIRIKLYACCGLVHGPVEAIENLQGRYPELLNRANLSNIRHVHVQLSTASNSHCGWIPEERPISSIAGQMSVAYILAVQLVDQQCLLSQFSEFDDNLERPEVWDLARKVTSSQSEEFDQDGNCLSAGRVRIEFNDGSSITESVEKPLGVKEPMPNERILHKYRTLAGSVTDESRVKEIEDLVLGLDRLTDISPLLELLNCPVKSPLGI

SEQ ID NO: 4 - DNA, 1476 bp, Aspergillus terreus
atgaccaaacaatctgcggacagcaacgcaaagtcaggagttacgtccgaaatatgtcattgggcatccaacctggccactgacgacatcccttcggacgtattagaaagagcaaaataccttattctcgacggtattgcatgtgcctgggttggtgcaagagtgccttggtcagagaagtatgttcaggcaacgatgagctttgagccgccgggggcctgcagggtgattggatatggacagaaactggggcctgttgcagcagccatgaccaattccgctttcatacaggctacggagcttgacgactaccacagcgaagcccccctacactctgcaagcattgtccttcctgcggtctttgcagcaagtgaggtcttagccgagcagggcaaaacaatttccggtatagatgttattctagccgccattgtggggtttgaatctggcccacggatcggcaaagcaatctacggatcggacctcttgaacaacggctggcattgtggagctgtgtatggcgctccagccggtgcgctggccacaggaaagctcctcggtctaactccagactccatggaagatgctctcggaattgcgtgcacgcaagcctgtggtttaatgtcggcgcaatacggaggcatggtaaagcgtgtgcaacacggattcgcagcgcgtaatggtcttcttgggggactgttggcccatggtgggtacgaggcaatgaaaggtgtcctggagagatcttacggcggtttcctcaagatgttcaccaagggcaacggcagagagcctccctacaaagaggaggaagtggtggctggtctcggttcattctggcatacctttactattcgcatcaagctctatgcctgctgcggacttgtccatggtccagtcgaggctatcgaaaaccttcaggggagataccccgagctcttgaatagagccaacctcagcaacattcgccatgttcatgtacagctttcaacggcctcgaacagtcactgtggatggataccagaggagagacccatcagttcaatcgcagggcagatgagtgtcgcatacattctcgccgtccagctggtcgaccagcaatgtcttttgtcccagttttctgagtttgatgacaacctggagaggccagaagtttgggatctggccaggaaggttacttcatctcaaagcgaagagtttgatcaagacggcaactgtctcagtgcgggtcgcgtgaggattgagttcaacgatggttcttctattacggaaagtgtcgagaagcctcttggtgtcaaagagcccatgccaaacgaacggattctccacaaataccgaacccttgctggtagcgtgacggacgaatcccgggtgaaagagattgaggatcttgtcctcggcctggacaggctcaccgacattagcccattgctggagctgctgaattgccccgtgaaatcgccccttggtatataa

SEQ ID NO: 5 - protein, 492 aa, Aspergillus terreus
MTKQSADSNAKSGVTSEICHWASNLATDDIPSDVLERAKYLILDGIACAWVGARVPWSEKYVQATMSFEPPGACRVIGYGQKLGPVAAAMTNSAFIQATELDDYHSEAPLHSASIVLPAVFAASEVLAEQGKTISGIDVILAAIVGFESGPRIGKAIYGSDLLNNGWHCGAVYGAPAGALATGKLLGLTPDSMEDALGIACTQACGLMSAQYGGMVKRVQHGFAARNGLLGGLLAHGGYEAMKGVLERSYGGFLKMFTKGNGREPPYKEEEVVAGLGSFWHTFTIRIKLYACCGLVHGPVEAIENLQGRYPELLNRANLSNIRHVHVQLSTASNSHCGWIPEERPISSIAGQMSVAYILAVQLVDQQCLLSQFSEFDDNLERPEVWDLARKVTSSQSEEFDQDGNCLSAGRVRIEFNDGSSITESVEKPLGVKEPMPNERILHKYRTLAGSVTDESRVKEIEDLVLGLDRLTDISPLLELLNCPVKSPLGIK

SEQ ID NO: 6 - DNA, 1479 bp, Aspergillus terreus
atgaccaaacaatctgcggacagcaacgcaaagtcaggagttacgtccgaaatatgtcattgggcatccaacctggccactgacgacatcccttcggacgtattagaaagagcaaaataccttattctcgacggtattgcatgtgcctgggttggtgcaagagtgccttggtcagagaagtatgttcaggcaacgatgagctttgagccgccgggggcctgcagggtgattggatatggacagaaactggggcctgttgcagcagccatgaccaattccgctttcatacaggctacggagcttgacgactaccacagcgaagcccccctacactctgcaagcattgtccttcctgcggtctttgcagcaagtgaggtcttagccgagcagggcaaaacaatttccggtatagatgttattctagccgccattgtggggtttgaatctggcccacggatcggcaaagcaatctacggatcggacctcttgaacaacggctggcattgtggagctgtgtatggcgctccagccggtgcgctggccacaggaaagctcctcggtctaactccagactccatggaagatgctctcggaattgcgtgcacgcaagcctgtggtttaatgtcggcgcaatacggaggcatggtaaagcgtgtgcaacacggattcgcagcgcgtaatggtcttcttgggggactgttggcccatggtgggtacgaggcaatgaaaggtgtcctggagagatcttacggcggtttcctcaagatgttcaccaagggcaacggcagagagcctccctacaaagaggaggaagtggtggctggtctcggttcattctggcatacctttactattcgcatcaagctctatgcctgctgcggacttgtccatggtccagtcgaggctatcgaaaaccttcaggggagataccccgagctcttgaatagagccaacctcagcaacattcgccatgttcatgtacagctttcaacggcctcgaacagtcactgtggatggataccagaggagagacccatcagttcaatcgcagggcagatgagtgtcgcatacattctcgccgtccagctggtcgaccagcaatgtcttttgtcccagttttctgagtttgatgacaacctggagaggccagaagtttgggatctggccaggaaggttacttcatctcaaagcgaagagtttgatcaagacggcaactgtctcagtgcgggtcgcgtgaggattgagttcaacgatggttcttctattacggaaagtgtcgagaagcctcttggtgtcaaagagcccatgccaaacgaacggattctccacaaataccgaacccttgctggtagcgtgacggacgaatcccgggtgaaagagattgaggatcttgtcctcggcctggacaggctcaccgacattagcccattgctggagctgctgaattgccccgtgaaatcgcccctgggtataaaataa

SEQ ID NO: 7 - protein, 491 aa, Aspergillus terreus
MTKQSADSNAKSGVTSEICHWASNLATDDIPSDVLERAKYLILDGIACAWVGARVPWSEKYVQATMSFEPPGACRVIGYGQKLGPVAAAMTNSAFIQATELDDYHSEAPLHSASIVLPAVFAASEVLAEQGKTISGIDVILAAIVGFESGPRIGKAIYGSDLLNNGWHCGAVYGAPAGALATGKLLGLTPDSMEDALGIACTQACGLMSAQYGGMVKRVQHGFAARNGLLGGLLAHGGYEAMKGVLERSYGGFLKMFTKGNGREPPYKEEEVVAGLGSFWHTFTIRIKLYACCGLVHGPVEAIENLQGRYPELLNRANLSNIRHVHVQLSTASNSHCGWIPEERPISSIAGQMSVAYILAVQLVDQQCLLSQFSEFDDNLERPEVWDLARKVTSSQSEEFDQDGNCLSAGRVRIEFNDGSSITESVEKPLGVKEPMPNERILHKYRTLAGSVTDESRVKEIEDLVLGLDRLTDISPLLELLNCPVKSPFGI

SEQ ID NO: 8 - DNA, 1476 bp, Aspergillus terreus
atgaccaaacaatctgcggacagcaacgcaaagtcaggagttacgtccgaaatatgtcattgggcatccaacctggccactgacgacatcccttcggacgtattagaaagagcaaaataccttattctcgacggtattgcatgtgcctgggttggtgcaagagtgccttggtcagagaagtatgttcaggcaacgatgagctttgagccgccgggggcctgcagggtgattggatatggacagaaactggggcctgttgcagcagccatgaccaattccgctttcatacaggctacggagcttgacgactaccacagcgaagcccccctacactctgcaagcattgtccttcctgcggtctttgcagcaagtgaggtcttagccgagcagggcaaaacaatttccggtatagatgttattctagccgccattgtggggtttgaatctggcccacggatcggcaaagcaatctacggatcggacctcttgaacaacggctggcattgtggagctgtgtatggcgctccagccggtgcgctggccacaggaaagctcctcggtctaactccagactccatggaagatgctctcggaattgcgtgcacgcaagcctgtggtttaatgtcggcgcaatacggaggcatggtaaagcgtgtgcaacacggattcgcagcgcgtaatggtcttcttgggggactgttggcccatggtgggtacgaggcaatgaaaggtgtcctggagagatcttacggcggtttcctcaagatgttcaccaagggcaacggcagagagcctccctacaaagaggaggaagtggtggctggtctcggttcattctggcatacctttactattcgcatcaagctctatgcctgctgcggacttgtccatggtccagtcgaggctatcgaaaaccttcaggggagataccccgagctcttgaatagagccaacctcagcaacattcgccatgttcatgtacagctttcaacggcctcgaacagtcactgtggatggataccagaggagagacccatcagttcaatcgcagggcagatgagtgtcgcatacattctcgccgtccagctggtcgaccagcaatgtcttttgtcccagttttctgagtttgatgacaacctggagaggccagaagtttgggatctggccaggaaggttacttcatctcaaagcgaagagtttgatcaagacggcaactgtctcagtgcgggtcgcgtgaggattgagttcaacgatggttcttctattacggaaagtgtcgagaagcctcttggtgtcaaagagcccatgccaaacgaacggattctccacaaataccgaacccttgctggtagcgtgacggacgaatcccgggtgaaagagattgaggatcttgtcctcggcctggacaggctcaccgacattagcccattgctggagctgctgaattgccccgtgaaatcgccctttggtatataa

SEQ ID NO: 9 - DNA, 24 bp, Artificial Sequence
  PCR Primer
cagccatgaccaattccgctttca

SEQ ID NO: 10 - DNA, 24 bp, Artificial Sequence
  PCR Primer
aagacctcacttgctgcaaagacc

SEQ ID NO: 11 - DNA, 20 bp, Artificial Sequence
  PCR Primer
ttgtggagctgtgtatggcg

SEQ ID NO: 12 - DNA, 16 bp, Artificial Sequence
  PCR Primer
gttggcccatggtggg

SEQ ID NO: 13 - DNA, 20 bp, Artificial Sequence
  PCR Primer
catggctgctgcaacaggcc